(19) United States
(12) Patent Application Publication
(10) Pub. No.: US 2024/0120112 A1
(43) Pub. Date: Apr. 11, 2024

(54) BEAT CLUSTERING

(71) Applicant: BOSTON SCIENTIFIC CARDIAC DIAGNOSTICS, INC, Rochester, MN (US)

(72) Inventors: David R. Engebretsen, Cannon Falls, MN (US); Benjamin A. Teplitzky, Lakeville, MN (US); Jake Matras, Eagan, MN (US); Michael Thomas Edward McRoberts, Mazeppa, MN (US)

(21) Appl. No.: 18/376,071

(22) Filed: Oct. 3, 2023

Related U.S. Application Data
(60) Provisional application No. 63/413,442, filed on Oct. 5, 2022.

(51) Int. Cl. G16H 50/70

(52) U.S. Cl. CPC G16H 50/70

(57) ABSTRACT

A method includes associating beats with respective initial beat classifications using a trained machine learning model and based on electrocardiogram data. Using an encoder machine learning model, latent space representations of the electrocardiogram data are generated for beats associated with the initial beat classifications. Similar shaped beats are associated with each other based on the latent space representations.

### CROSS REFERENCE TO RELATED APPLICATION

This application claims priority to Provisional Application No. 63/413,442, filed Oct. 5, 2022, which is herein incorporated by reference in its entirety.

### TECHNICAL FIELD

The present disclosure relates to devices, methods, and systems for analyzing cardiac activity.

### BACKGROUND

Monitoring devices for collecting biometric data are becoming increasingly common in diagnosing and treating medical conditions in patients. For example, mobile devices can be used to monitor cardiac data in a patient. This cardiac monitoring can empower physicians with valuable information regarding the occurrence and regularity of a variety of heart conditions and irregularities in patients. Classifying individual heartbeats can help accurately identify and classify cardiac events such as abnormal cardiac rhythms so that critical alerts can be provided to patients, physicians, or other care providers and patients can be treated.

### SUMMARY

In Example 1, a method includes associating, using a trained machine learning model and based on electrocardiogram (ECG) data, beats with respective initial beat classifications. The initial beat classifications including a first classification and a second classification. The method further includes generating, using a first encoder machine learning model, first latent space representations of the ECG data for beats associated with the first classification. The method further includes generating, using a second encoder machine learning model, second latent space representations of the ECG data for beats associated with the second classification. Similar shaped beats are associated with each other based on the first latent space representations and the second latent space representations.
In Example 2, the method of Example 1, further including: generating, using the trained machine learning model, individual clips of the ECG data, the individual clips each representing a separate beat.
In Example 3, the method of Example 2, wherein the individual clips comprise an individual T-wave for each beat.
In Example 4, the method of any of Examples 2 and 3, further including: generating the first latent space representations based on the individual clips associated with the first classification; and generating the second latent space representations based on the individual clips associated with the second classification.
In Example 5, the method of any of the preceding Examples, wherein the first latent space representations include 8-15 datapoints for each beat associated with the first classification, and wherein the second latent space representations include 8-15 datapoints for each beat associated with the second classification.
In Example 6, the method of any of the preceding Examples, wherein the ECG data of each beat comprises a first number of datapoints, wherein a number of datapoints of the first latent space representations comprises 1-2% of the first number of datapoints.
In Example 7, the method of any of the preceding Examples, wherein the associating similar shaped beats includes assigning each group of similar shaped beats a same value.
In Example 8, the method of any of the preceding Examples, wherein the associating similar shaped beats includes grouping the similar shaped beats using a k-means clustering algorithm.
In Example 9, the method of any of the preceding Examples, wherein the first encoder machine learning model and the second machine learning model are neural networks trained using an imbalanced encoder and decoder.
In Example 10, the method of Example 9, wherein the encoder includes more layers of nodes than the decoder.
In Example 11, the method of any of the preceding Examples, wherein the first encoder machine learning model and the second machine learning model are deep neural networks.
In Example 12, the method of any of the preceding Examples, wherein the first classification is a normal beat classification, wherein the second classification is a ventricular beat classification, wherein the initial beat classification further includes a supraventricular beat classification.
In Example 13, a computer program product comprising instructions to cause one or more processors to carry out the steps of the method of Examples 1-12.
In Example 14, a computer-readable medium having stored thereon the computer program product of Example 13.
In Example 15, a computer comprising the computer-readable medium of Example 14.
In Example 16, a method includes associating beats with respective initial beat classifications using a trained machine learning model and based on electrocardiogram data. Using an encoder machine learning model, latent space representations of the electrocardiogram data are generated for beats associated with the initial beat classifications. Similar shaped beats are associated with each other based on the latent space representations.
In Example 17, the method of Example 16, wherein the one or more encoder machine learning models includes a first encoder machine learning model and a second encoder machine learning model, wherein the initial beat classifications include a first classification and a second classification, wherein the latent space representations include first latent space representations for beats associated with the first classification and second latent space representations for beats associated with the second classification. The method further includes: generating, using the first encoder machine learning model, the first latent space representations; and generating, using the second encoder machine learning model, the second latent space representations.
In Example 18, the method of Example 17, further including: generating, using the trained machine learning model, individual clips of the ECG data, the individual clips each representing a separate beat.
In Example 19, the method of Example 18, wherein the individual clips comprise an individual T-wave for each beat.
In Example 20, the method of Example 18, further including: generating the first latent space representations based on the individual clips associated with the first classification; and generating the second latent space representations based on the individual clips associated with the second classification.
In Example 21, the method of Example 18, wherein the first latent space representations include 8-15 datapoints for each beat associated with the first classification, wherein the second latent space representations include 8-15 datapoints for each beat associated with the second classification.
In Example 22, the method of Example 17, wherein the first classification is a normal beat classification, wherein the second classification is a ventricular beat classification, wherein the initial beat classifications further include a supraventricular beat classification.
In Example 23, the method of claim 16, wherein the ECG data of each beat comprises a first number of datapoints, wherein a number of datapoints of the latent space representations comprises 1-2% of the first number of datapoints.
In Example 24, the method of Example 16, wherein the associating similar shaped beats includes grouping the similar shaped beats using a k-means clustering algorithm.
In Example 25, the method of Example 16, wherein the associating similar shaped beats includes assigning each group of similar shaped beats a same value.
In Example 26, the method of Example 25, further including: receiving—by a computing system—the ECG data, the initial beat classifications, and the values assigned to the beats; displaying the ECG data in a user interface (UI); receiving a command to change at least some of the initial beat classifications to a subsequent beat classifications; and automatically modifying the values to a different value associated with the subsequent beat classifications.
In Example 27, a system includes a server with: a first trained machine learning model programmed to generate first latent space representations of beats associated with a first beat classification, a second trained machine learning model programmed to generate second latent space representations of beats associated with a second beat classification, a third trained machine learning model programmed to generate third latent space representations of beats associated with a third beat classification, and one or more processors programmed to apply a clustering algorithm to the first, second, and third latent space representations to associating similar shaped beats with each other.
In Example 28, the system of Example 27, wherein ECG data of each beat comprises a first number of datapoints, wherein a number of datapoints of the first, second, and third latent space representations comprises 1-2% of the first number of datapoints.
In Example 29, the system of Example 27, wherein the first, second, and third trained machine learning models are deep learning neural networks.
In Example 30, the system of Example 27, wherein the clustering algorithm is a k-means clustering algorithm.
In Example 31, the system of Example 27, wherein the first beat classification is a normal beat classification, wherein the second beat classification is a ventricular beat classification, wherein the third beat classification is a supraventricular beat classification.
In Example 32, the system of Example 27, wherein first, second, and third trained machine learning models are neural networks trained using an imbalanced encoder and decoder.
In Example 33, a method for training an encoder neural network includes inputting data into the encoder neural network, which includes a first number of layers of nodes. The method further includes generating latent space representations of the data by the encoder neural network and inputting the latent space representations into a decoder with a second number of layers of nodes, which is less than the first number. The method further includes training the encoder neural network based on the outputs of the decoder responsive to the inputting the latent space representations.
In Example 34, the method of Example 33, wherein the data is ECG data and further comprising: inputting metadata associated with the ECG data into the encoder neural network.
In Example 35, the method of Example 33, wherein the first number is 4-8 times the second number.

While multiple instances are disclosed, still other instances of the present disclosure will become apparent to those skilled in the art from the following detailed description, which shows and describes illustrative instances of the disclosure. Accordingly, the drawings and detailed description are to be regarded as illustrative in nature and not restrictive.

### BRIEF DESCRIPTION OF THE DRAWINGS

FIG. 1 shows a cardiac monitoring system, in accordance with certain instances of the present disclosure.

FIG. 2 shows a server, remote computer, and user interface, in accordance with certain instances of the present disclosure.

FIG. 3 shows an example of beats that have been grouped together and displayed in a user interface, in accordance with certain instances of the present disclosure.

FIGS. 4-8 show various views of a report building user interface, in accordance with certain instances of the present disclosure.

FIGS. 9 and 10 show block diagrams depicting an illustrative methods, in accordance with certain instances of the disclosure.

FIG. 11 is a block diagram depicting an illustrative computing device, in accordance with instances of the disclosure.

While the disclosed subject matter is amenable to various modifications and alternative forms, specific instances have been shown by way of example in the drawings and are described in detail below. The intention, however, is not to limit the disclosure to the particular instances described. On the contrary, the disclosure is intended to cover all modifications, equivalents, and alternatives falling within the scope of the disclosure as defined by the appended claims.

### DETAILED DESCRIPTION

The present disclosure relates to devices, methods, and systems for classifying heartbeats (hereinafter “beats” for brevity) and grouping together similarly shaped beats in clusters. Such classification and clustering facilitates analysis of cardiac activity.

Electrocardiogram (ECG) data of a patient can be used to identify whether the patient has experienced a cardiac event and what type of cardiac event occurred. One input to determining the type of cardiac event includes the types (or classifications) of beats experienced during the cardiac event. For example, an ECG analysis system may automatically determine that a certain type of cardiac event occurred based on—among other things—how the system classified the beats occurring during the event. However, if the beats were initially misclassified, the determined type of cardiac event may also be misclassified and therefore may then need to be reclassified. Instances of the present disclosure are accordingly directed to systems, methods, and devices for classifying and grouping beats and additionally for facilitating analysis and reclassification of beats.

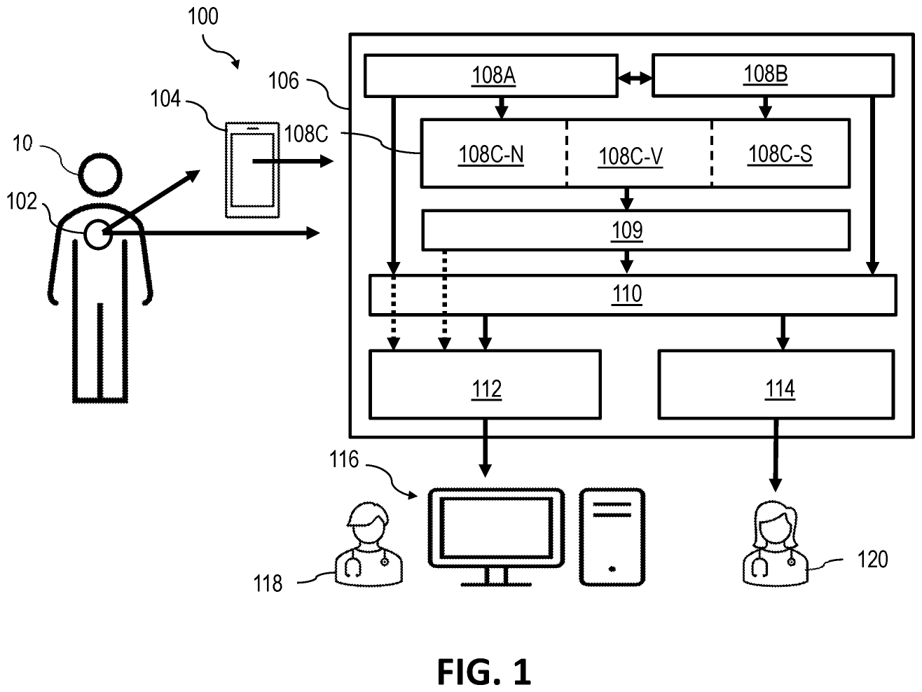

FIG. 1 illustrates a patient 10 and an example system 100. The system 100 includes a monitor 102 attached to the patient 10 or implanted in the patient 10 (e.g., pacemaker, ICD, CRT, or ICM) to detect cardiac activity of the patient 10. The monitor 102 may produce electric signals that represent the cardiac activity in the patient 10. For example, the monitor 102 may detect the patient's heart beating (e.g., using infrared sensors, electrodes, heart sounds) and convert the detected heartbeat into electric signals representing ECG data. In certain instances, the monitor 102 stores the ECG data of a patient study (e.g., one or more days of ECG data), after which the ECG data is transmitted to another device or system such as a server. Additionally or alternatively, the monitor 102 transmits the ECG data to a mobile device 104 (e.g., a mobile phone). In such instances, the mobile device 104 can include a program (e.g., mobile phone application) that receives, processes, and analyzes the ECG data. For example, the program may analyze the ECG data and detect or flag cardiac events (e.g., periods of irregular cardiac activity) contained within the ECG data. The mobile device 104 can periodically transmit chunks of the ECG data to another device or system such as a server, which can process, append together, and archive the chunks of the ECG data and metadata (e.g., time, duration, detected/flagged cardiac events) associated with the chunks of ECG data. In certain instances, the monitor 102 may be programmed to transmit the ECG data directly to the other device or system without utilizing the mobile device 104. Also, the monitor 102 and/or the mobile device 104 includes a button or touch-screen icon that allows the patient 10 to initiate an event. Such an indication can be recorded and communicated to the other device or system. In other instances involving multi-day studies, the ECG data and associated metadata are transmitted in larger chunks (e.g., an entire study's worth of ECG data).

### Cardiac Event Server

The ECG data (and associated metadata, if any) is transmitted to and stored by a cardiac event server 106 (hereinafter “the server 106” for brevity). The server 106 includes multiple models, platforms, layers, or modules that work together to process and analyze the ECG data such that cardiac events can be detected, filtered, prioritized, and ultimately reported to a patient's physician for analysis and treatment. In the example of FIG. 1, the server 106 includes one or more machine learning models 108A, 108B, and 108C, a clustering algorithm module 109, a cardiac event router 110, a report platform 112, and a notification platform 114. Although only one server 106 is shown in FIG. 1, the server 106 can include multiple separate physical servers, and the various models/platforms/modules/layers can be distributed among the multiple servers. Each of the models/platforms/modules/layers can represent separate programs, applications, and/or blocks of code where the output of one of the models/platforms/modules/layers is an input to another of the models/platforms/modules/layers. Each of the models/platforms/modules/layers can use application programming interfaces to communicate between or among the other models/platforms/modules/layers as well as systems and devices external to the server 106.

In certain instances, once the ECG data is processed by the machine learning models 108A-C and the clustering algorithm module 109, the ECG data (and associated metadata) is made available for the report platform 112. As will be described in more detail below, the report platform 112 can be accessed by a remote computer 116 (e.g., client device such as a laptop, mobile phone, desktop computer, and the like) by a user at a clinic or lab 118. In other instances, the cardiac event router 110 is used to determine what platform further processes the ECG data based on the classification associated with the cardiac event. For example, if the identified cardiac event is critical or severe, the cardiac event router 110 can flag or send the ECG data, etc., to the notification platform 114. The notification platform 114 can be programmed to send notifications (along with relevant ECG data and associated metadata) immediately to the patient's physician/care group remote computer 116 and/or to the patient 10 (e.g., to their computer system, e-mail, mobile phone application).

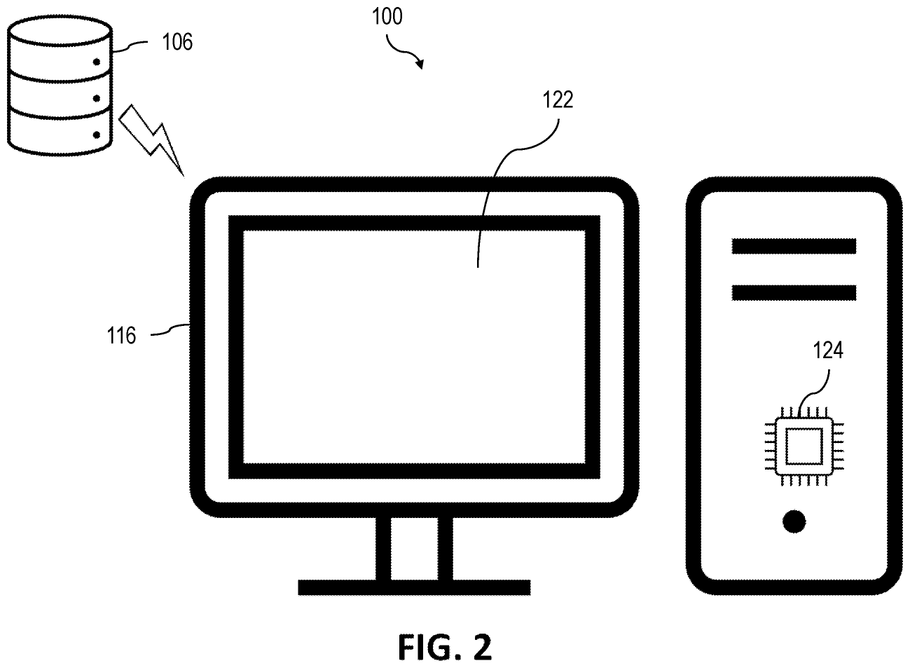

FIG. 2 shows the server 106 communicatively coupled (e.g., via a network) to the remote computer 116. In the example of FIG. 2, the remote computer 116 includes a monitor showing a user interface 122 (hereinafter “the UI 122” for brevity) that displays features of the report platform 112 hosted by the server 106. The UI 122 includes multiple pages or screens for tracking and facilitating analysis of patient ECG data.

In certain instances, the report platform 112 is a software-as-a-service (SaaS) platform hosted by the server 106. To access the report platform 112, a user (e.g., a technician) interacts with the UI 122 to log into the report platform 112 via a web browser such that the user can use and interact with the report platform 112.

### Machine Learning Models

The server 106 applies the one or more machine learning models 108A-C to the ECG data to analyze and classify the beats and cardiac activity of the patient 10.

As described in more detail below, the first and second machine learning models 108A and 108B are programmed to—among other things—compare the ECG data to labeled ECG data to determine which labeled ECG data the ECG data most closely resembles. The labeled ECG data may identify a particular cardiac event—including but not limited to ventricular tachycardia, bradycardia, atrial fibrillation, pause, normal sinus rhythm, or artifact/noise—as well as particular beat classifications—including but not limited to ventricular, normal, or supraventricular. In addition to identifying beat classifications and event classifications (and generating associated metadata), the first and second machine learning models 108A and 108B can determine and generate metadata regarding heart rates, duration, and beat counts of the patient 10 based on the ECG data. As specific examples, the first and/or the second machine learning models 108A and 108B can identify the beginning, center, and end of individual beats (e.g., individual T-waves) such that individual beats can be extracted from the ECG data. Each individual beat can be assigned a value (e.g., a unique identifier) such that individual beats can be identified and associated with metadata throughout processing and analyzing the ECG data.

The ECG data (e.g., ECG data associated with individual beats) as well as certain outputs of the first and second machine learning models 108A and 108B can be inputted to the third machine learning model 108C. Although two machine learning models are shown and described, a single machine learning model could be used to generate the metadata described herein, or additional machine learning models could be used.

The first and second machine learning models 108A and 1088 can include the neural networks described in Ser. No. 16/695,534, which is hereby incorporated by reference in its entirety. The first neural network can be a deep convolutional neural network and the second neural network is a deep fully-connected neural network—although other types and combinations of machine learning models can be implemented. The first machine learning model 108A receives one or more sets of beats (e.g., beat trains with 3-10 beats) which are processed through a series of layers in the deep convolutional neural network. The series of layers can include a convolution layer to perform convolution on time series data in the beat trains, a batch normalization layer to normalize the output from the convolution layer (e.g., centering the results around an origin), and a non-linear activation function layer to receive the normalized values from the batch normalization layer. The beat trains then pass through a repeating set of layers such as another convolution layer, a batch normalization layer, a non-linear activation function layer. This set of layers can be repeated multiple times.

The second machine learning model 108B receives RR-interval data (e.g., time intervals between adjacent beats) and processes the RR-interval data through a series of layers: a fully connected layer, a non-linear activation function layer, another fully connected layer, another non-linear activation function layer, and a regularization layer. The output from the two paths is then provided to the fully connected layer. The resulting values are passed through a fully connected layer and a softmax layer to produce probability distributions for the classes of beats.

The third machine learning model 108C (e.g., one or more trained encoder machine learning models) is programmed to generate latent space representations of the ECG data such that the ECG data is represented by fewer datapoints than the original ECG data. The latent space representations can be used as an approximation of the original raw ECG data for each beat. Although the inputs to the third machine learning model 108C are described as (1) the ECG data such as sets of individual T-waves and (2) certain outputs of the first and second machine learning models 108A and 108B, the third machine learning model 108C could be programmed to generate the latent space representations without requiring input from the first and/or second machine learning models 108A, 108B.

In certain instances, instead of a single third machine learning model 108C, the server 106 includes a separate machine learning model for each type of beat classification (e.g., normal beats, ventricular beats, and supraventricular beats). For example, as shown in FIG. 1, the server 106 may include three third machine learning models (108C-N, 108C-V, and 108C-S) instead of a single third machine learning model. In certain instances, beats that were not initially classified (e.g., unclassified beats) can be processed either by a different third machine learning model or can skip the step of generating latent space representations and being clustered with similar shaped beats.

In the example of FIG. 1, one machine learning model 108C-N is used for beats classified as normal beats, another machine learning model 108C-V is used for beats classified as ventricular beats, and another machine learning model 108C-S is used for beats classified as supraventricular beats. As such, only ECG data (e.g., T-waves) of beats initially classified as normal beats by the first and/or second machine learning models 108A, 108B—as well as metadata generated by such machine learning models—are inputted to the machine learning model 108C-N, and so on. It has been found that using machine learning models trained to focus on analyzing only certain types of beats can improve performance of the third machine learning models compared to using a single third machine learning model. Further, processing the ECG data in parallel using three machine learning models can decrease the time needed to generate the latent space representations. In certain instances, a single study may contain hundreds of thousands to millions of individual beats.

Each third machine learning model (108C-N, 108C-V, 108C-S) receives ECG data associated with individual beats (e.g., an individual clip of ECG data for each beat) and generates latent space representations of such ECG data. For example, each individual beat is processed by one of the third machine learning models—depending on each individual beat's classification—such that the ECG data is distilled down to (or represented by) a small number of individual data points. Raw ECG data of an individual beat can include 500 or so datapoints, and each third machine learning model can distill the ECG data for a given beat into 4-16 datapoints. Put another way, each third machine learning model can generate latent space representations comprising 4-16 datapoints for a given beat. This range has been found to balance accuracy of beat representation and effectiveness of clustering (described further below). In certain instances, the latent space representations comprise 7, 8, or 9 (e.g., 7-9) datapoints fora given beat The latent space representations comprise 1-2% of datapoints compared to the raw ECG data for each beat. Each latent space can be represented by a vector (e.g., a latent vector).

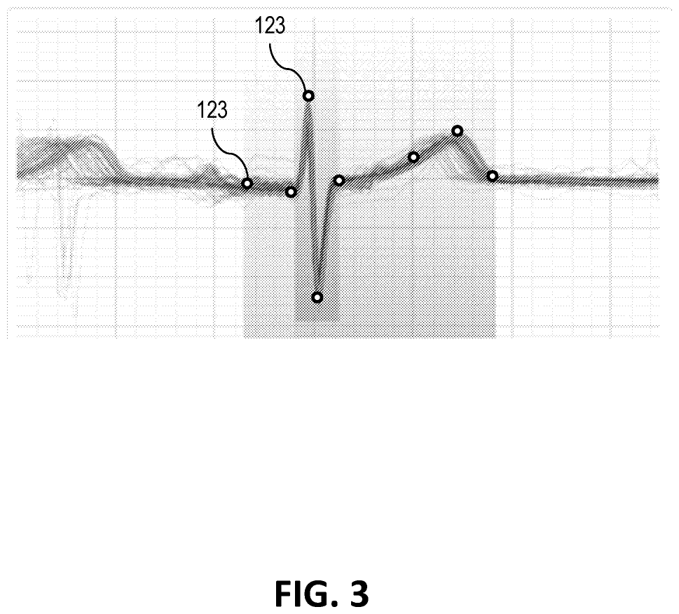

The resulting datapoints are representations of an amplitude of the ECG signal at different relative points in time. These limited datapoints are datapoints that the trained machine learning models generate such that different beat shapes can be identified and similar shaped beats can be grouped together. Put another way, these datapoints may be those that are the most likely to be helpful in distinguishing among beat shapes. The third machine learning models can leave out representations of datapoints that are less likely to help distinguish among individual beats. FIG. 3 shows an example set of beats that have been grouped or clustered together and also shows non-limiting examples of points 123 within a beat's ECG signal that may be useful for distinguishing among beat shapes. For example, the points 123 can be located at the beginning and end of each beat, apexes (e.g., QRS peak), nadirs, etc.

In the example of FIG. 1, the third machine learning models (108C-N, 108C-V, 108C-S) generate respective separate latent space representations for sets of beats initially classified as normal beats, ventricular beats, and supraventricular beats. In certain instances, beats that could not be initially classified (or ECG data containing artifacts due to noise) are not processed by any of the third machine learning models.

The output(s) of the third machine learning model(s) 108C are processed by a clustering algorithm module 109. The clustering algorithm module 109 receives the latent space representations of individual beats and is programmed to associate similar shaped beats into different groups. FIG. 3 shows an example set of beats that have been grouped or clustered together. As shown in FIG. 3, ECG waveforms of individual beats (e.g., T-waves) are superimposed on each other. Each cluster or group can include hundreds or thousands of beats that have been grouped together by the clustering algorithm module 109. As can be seen, the beats all have a similar profile relative to each other. Each beat is aligned with the other beats to have respective QRS peaks centered on the graph.

In certain instances, the clustering algorithm module 109 is programmed to apply a clustering algorithm such as the k-means clustering algorithm or a derivation or variation thereof to the latent space representations. In certain instances, the same clustering algorithm module 109 and the same algorithm is used to process the latent space representations of each of the third machine learning models (108C-N, 108C-V, 108C-S). In certain instances, the output of the clustering algorithm module 109 includes assigning a value (e.g., an identifier such as a number) to each beat that is indicative of the group selected by the clustering algorithm module 109. For example, if the clustering algorithm module 109 clusters the beats into eight different groups, then all beats selected to be in the first group may be assigned a value of “1” and all beats selected to be in the second group may be assigned a value of “2” and so on. Other types of values can be used. These group values can be added to the metadata associated with each beat.

As described in more detail below, the groups of beats are ultimately presented to an end user in an ECG analysis tool and used for efficient review of a large amount of ECG data (e.g., one or more days' of ECG data).

### Report Build Page

The server 106 (e.g., via programming associated with the report platform 112) can start a process for sending data to the remote computer 116. This data includes the ECG data and metadata associated with the ECG data. Accessing, processing, and displaying one or more days' worth of ECG data and metadata can consume a large amount of computing resources, network bandwidth resources, and human resources. To help alleviate burdens on these resources, the server 106 (e.g., via the report platform 112) can selectively transmit packages of ECG data and metadata to the remote computer 116.

The initial packages of data can include: (1) short strips (e.g., 60-second strips) of ECG data surrounding detected cardiac events, (2) metadata associated with the strips (e.g., beat metadata), and (3) executable code (e.g., JavaScript code). In certain instances, only the ECG data associated with highest priority cardiac events are initially transferred. After the initial packages of data are transmitted from the server to the remote computer 116, additional packages of data can be transmitted in response to selections made by the user in the UI 122.

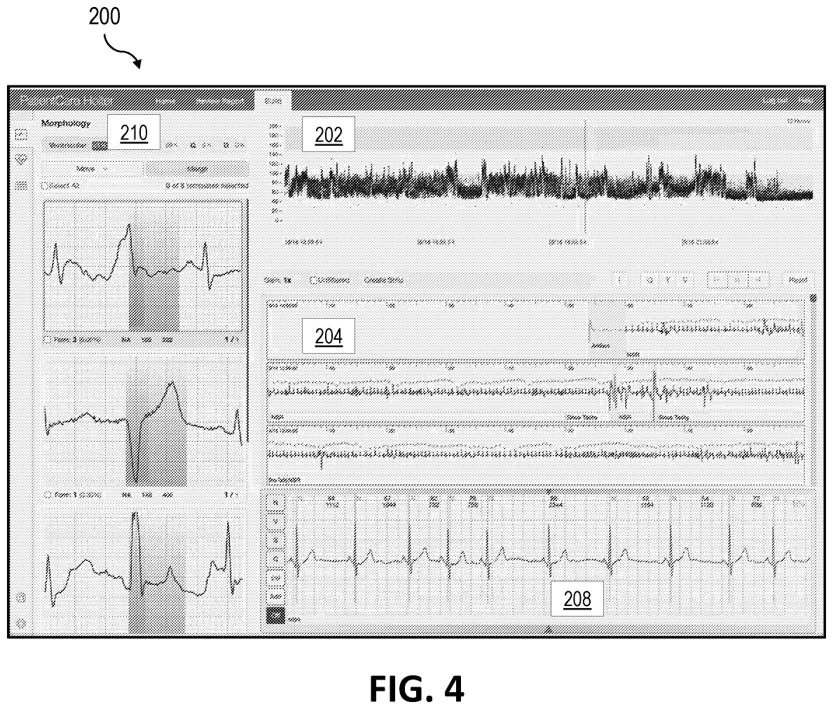

With these initial packages of data, the user has access (via the remote computer 116 and the UI 122) to a report build page 200 shown in FIGS. 4-8. The report build page 200 facilitates analysis of cardiac events and metadata associated with the cardiac events. The pages are generated at the remote computer 116 based on the packages of data and they are selectively displayed via the UI 122. As will be described in more detail below, as the user interacts with the report build page 200, the metadata is updated in real time at the remote computer 116.

FIG. 4 shows a screenshot of the report build page 200. The report build page 200 is used by a user to review and analyze a patient's ECG data and metadata. The report build page 200 includes multiple windows for displaying data, plots, icons, links, markers, indicators, and the like.

Window 202 displays a heart rate plot of multiple days' worth of ECG data. This window 202 provides an initial visual insight into which periods of time appear to contain abnormal heart rate activity. In the example of FIG. 4, the window 202 displays four days of ECG data, although shorter or longer time periods could be displayed by default or changed by the user.

Window 204 allows the user to view a shorter plot of ECG data. For example, the window 204 may display ECG data associated with a detected cardiac event along with ECG data preceding and following the detected event. This window 204 provides visual insight into the onset of a detected event and whether the detected event is perhaps an artifact, follow-on event, etc. As the user scrolls through the window 204, the window 202 can display an indicator (e.g., a vertical line) showing the location of the ECG data of window 204 within the heart rate plot of window 202.

Window 208 shows a plot of ECG data (e.g., approximately 10 beats) that is shorter than the plots of windows 202 and 204. Window 208 displays a closer-up view of a portion of the ECG data of windows 202 and 204. The user can use window 204 to select which shorter set of ECG data is displayed in the window 208. Each of the windows 202, 204, and 208 can include markers, indicators, icons, etc., to visually note the location of detected cardiac events within the strips of ECG data.

### Beat Morphology

To the left of the report build page 200 in FIG. 4 is a beat morphology window 210 (hereinafter “the morphology window 210”), which includes multiple individual sub-windows that display plots of one or more individual beats. The user can use the morphology window 210 to select beats, which can result in updating the windows 202, 204, and 208. For example, the windows 202, 204, and 208 can be updated to show ECG data surrounding the beat selected in the morphology window 210.

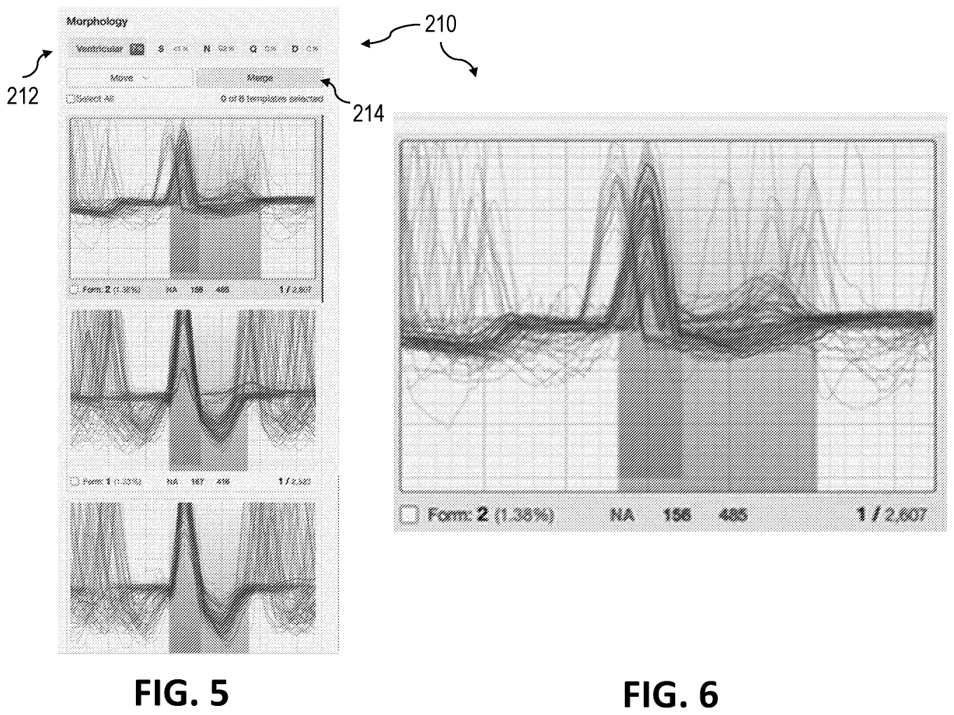

FIGS. 5 and 6 show closer-up alternative views of the morphology window 210. FIG. 5 shows the morphology window 210 as displaying multiple sub-windows. Each sub-window can display multiple plots of beats that have been characterized (e.g., by the clustering algorithm module 109) as having similar shaped (or morphology). The user can select the type of cardiac event (e.g., atrial fibrillation, ventricular tachycardia) from a menu button 212 to display groups of beats associated with the selected cardiac event in the sub-window.

The user can then select individual or sets of beat plots in the sub-window and, if desired, change the type of cardiac event the selected beats are associated with or the beat classification. Additionally, instead of selecting individual beats, the user can select all beats associated with a given cardiac event and change that given cardiac event (or beat classification) to a different type of cardiac event (or beat classification). Because the machine learning model 108A assigns each beat with an initial beat classification, the report build page 200 can be used to make mass updates to the metadata associated with similarly characterized beats. For example, in FIG. 6, the cardiac event type associated with the sub-window is shown as being associated with 2,607 beats. Using the morphology window 210 of the report build page 200, the user can recharacterize each of those 2,607 beats with one operation. The user can also merge sub-windows using a merge button 214 such that the beats in the two or more sub-windows are characterized with the same cardiac event type.

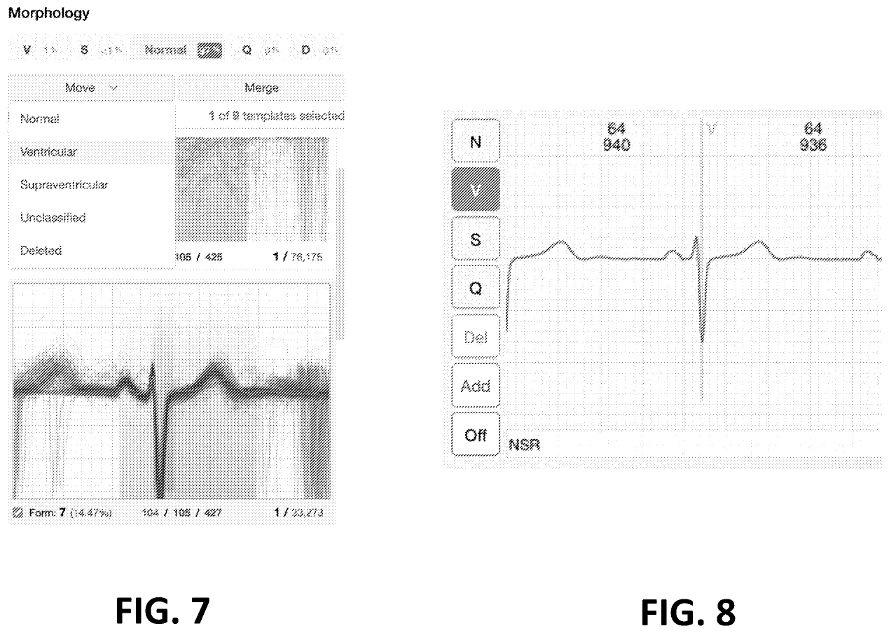

FIGS. 7 and 8 show other views of how a user can utilize a UI to make changes to beats. In FIG. 7, the user can select a window showing a grouping of similarly classified or grouped beats and then select a dropdown menu of options for reclassifying the selected grouping of beats. As shown in FIG. 7, potential options for classification include normal beats, ventricular beats, supraventricular beats, and unclassified beats. FIG. 8 shows another view where a user can select a beat or set of beats and use buttons to change the classification associated with the selected beat(s).

Using one or more of the approaches described above, metadata for thousands to hundreds of thousands (or millions, for long studies) of beats can be updated en masse through the UI. Because a set of ECG data may represent tens of thousands, hundreds of thousands, or even millions of individual beats, this ability to make mass updates to beats saves the user time in analyzing ECG data and, ultimately, building a report.

To save processing and network resources and to allow these changes to metadata to occur in real-time, the calculations and changes to the cardiac event classifications and the automatic updates to the beat classifications can be carried out locally on the remote computer 116—as opposed to sending data back and forth between the server 106 and the remote computer 116. For example, the reclassifications can be carried out using cache memory 124 (shown in FIG. 1) and processing capabilities (e.g., one or more microprocessors) of the remote computer 116. To enable local processing and updating, the report platform 112 can send the remote computer 116 code to execute locally. This code uses (or operates on) the outputs of the machine learning model 108 such as the beat classifications and rhythm classifications (as opposed to the underlying or raw ECG data), which reduces the computational resources needed to process the changes made by user locally at the remote computer 116. In certain embodiments, this code is executed by an internet browser operating on the remote computer 116.

In certain instances, once a final report is built and complete, the remote computer 116 can send any changes to the metadata (e.g., the subsequent beat classifications and the subsequent rhythm classifications) to the server 106 and its database. The server 106 can then replace the metadata initially created by the machine learning model (and saved to the database) with the metadata generated by the remote computer while the user was reviewing and editing the metadata. As such, if the ECG and metadata need to be accessed again, the server's database has the most recent version of the metadata. Further, the machine learning models 108 may be further trained on the metadata generated by the user at the remote computer.

### Methods

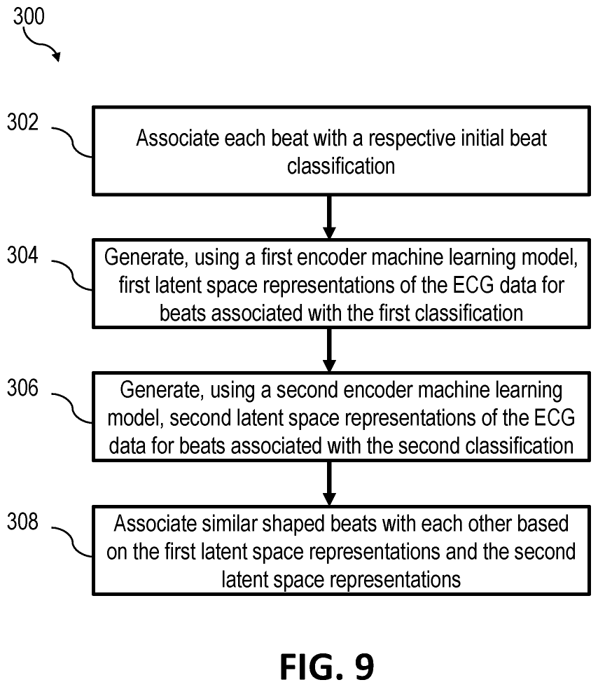

FIG. 9 shows an outline of steps of a method 300 for clustering groups of beats. The method 300 can include various steps and/or functions described above and is not limited necessarily to the steps shown in FIG. 9.

The method 300 includes using one or more trained machine learning models to associate beats with respective initial beat classifications (block 302 of FIG. 9). The initial beat classifications can be selected from a list of classifications, which may include a first classification (e.g., normal beat) and a second classification (e.g., ventricular beat).

The method 300 further includes generating, using a first encoder machine learning model, first latent space representations of the ECG data for beats associated with the first classification (block 304 of FIG. 9). The method 300 further includes generating, using a second encoder machine learning model, second latent space representations of the ECG data for beats associated with the second classification (block 306 of FIG. 9).

The method 300 further includes associating similar shaped beats with each other based on the first latent space representations and the second latent space representations (block 308 of FIG. 9).

### Training

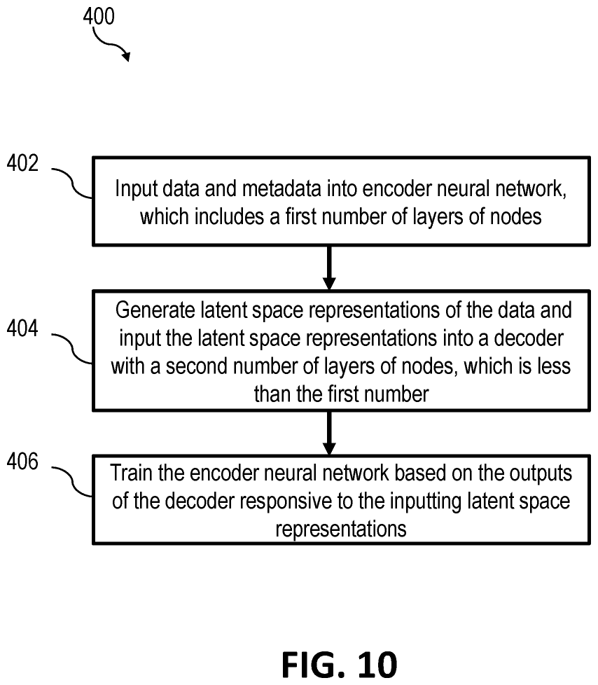

FIG. 10 shows an outline of steps of a method 400 for training the machine learning model 108C (including models 108C-N, 108C-V, 108C-S). In certain instances, the machine learning model 108 is an autoencoder neural network (e.g., an autoencoder DNN). Autoencoder neural networks can be trained to take a large set of input data (e.g., hundreds of thousands or millions of beats worth of ECG data) and extract features into much smaller latent space representations which can be used as an approximation of the original input data.

The autoencoder neural network can be trained using an imbalanced or asymmetric approach. For example, the machine learning model(s) 108C can be an autoencoder that is trained with a decoder that is a simpler neural network compared to the neural network of the autoencoder. More specifically, the autoencoder neural network can comprise more layers of nodes (e.g., hidden layers or intermediate layers of nodes) than the number of layers of nodes of the decoder. In some instances, the autoencoder neural network includes 4-8 times the number of layers as the decoder. For example, the autoencoder could include 12 layers while the decoder comprises 2 layers.

With the different number of respective layers in the autoencoder and decoder neural networks during training, the autoencoder neural network is forced to do more learning and generate better latent space representations that the weaker decoder can use for accurate decoding. For example, because the autoencoder neural network is trained by trying to match the output of the decoder to what is inputted to the autoencoder, the autoencoder is forced to train harder to provide quality latent space representations for the decoder. In certain instances, the autoencoder neural network is trained using ECG data from millions of individual beats and respective metadata. In such instances, the autoencoder neural network is trained using ECG data as well as outputs of trained machine learning models (e.g., models such as the machine learning models 108A and 108B). Further, in instances with multiple machine learning models 108C dedicated to processed certain types of beats, the machine learning models 108C can be trained only on ECG data associated with beats with a certain beat classification. Once the autoencoder neural network is trained, it can be implemented as the third machine learning model(s) 108C.

The method 400 includes inputting ECG data and metadata into encoder neural network, which includes a first number of layers of nodes (block 402 in FIG. 10). The ECG data can include individual clips of ECG data associated with respective beats, and the metadata can include outputs of one or more other machine learning models.

The method 400 further includes generating latent space representations of the ECG data by the encoder neural network and inputting the latent space representations into a decoder with a second number of layers of nodes, which is less than the first number (block 404 in FIG. 10).

The method 400 further includes training the encoder neural network based on the outputs of the decoder responsive to the inputting latent space representations (block 406 in FIG. 10).

Although the paragraphs in this section describe an encoder neural network used to generate latent space representations of beats, the described imbalanced or asymmetric training approach can be used for encoder neural networks used in different applications and with different types of input data. As such, the present disclosure describes approaches for training an encoder neural network for a wide variety of applications and data by utilizing an encoder neural network that has more layers of nodes that the decoder used during training. This approach forces the encoder neural network to work harder (and become better trained) compared to training approaches with encoders/decoders having the same number of layers of nodes.

### Computing Devices and Systems

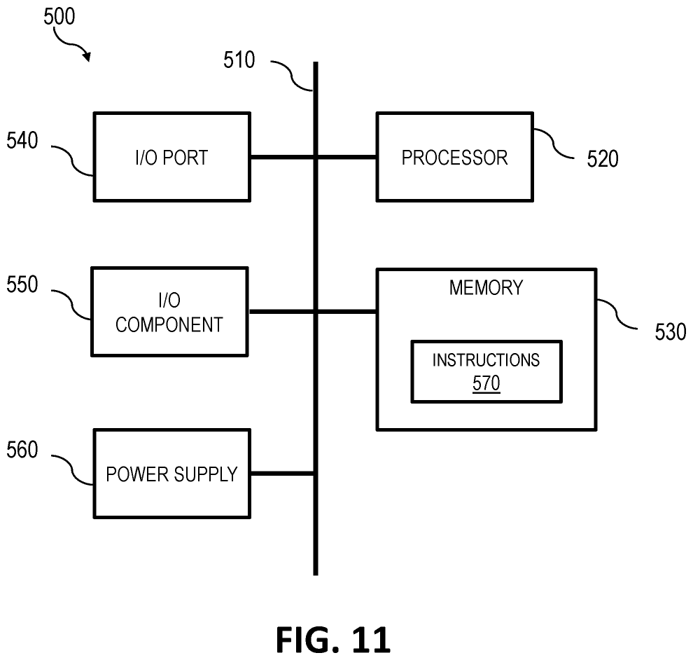

FIG. 11 is a block diagram depicting an illustrative computing device 500, in accordance with instances of the disclosure. The computing device 500 may include any type of computing device suitable for implementing aspects of instances of the disclosed subject matter. Examples of computing devices include specialized computing devices or general-purpose computing devices such as workstations, servers, laptops, desktops, tablet computers, hand-held devices, smartphones, general-purpose graphics processing units (GPGPUs), and the like. Each of the various components shown and described in the Figures can contain their own dedicated set of computing device components shown in FIG. 11 and described below. For example, the mobile device 104, the server 106, and the remote computer 116 can each include their own set of components shown in FIG. 11 and described below.

In instances, the computing device 500 includes a bus 510 that, directly and/or indirectly, couples one or more of the following devices: a processor 520, a memory 530, an input/output (I/O) port 540, an I/O component 550, and a power supply 560. Any number of additional components, different components, and/or combinations of components may also be included in the computing device 500.

The bus 510 represents what may be one or more busses (such as, for example, an address bus, data bus, or combination thereof). Similarly, in instances, the computing device 500 may include a number of processors 520, a number of memory components 530, a number of I/O ports 540, a number of I/O components 550, and/or a number of power supplies 560. Additionally, any number of these components, or combinations thereof, may be distributed and/or duplicated across a number of computing devices.

In instances, the memory 530 includes computer-readable media in the form of volatile and/or nonvolatile memory and may be removable, nonremovable, or a combination thereof. Media examples include random access memory (RAM); read only memory (ROM); electronically erasable programmable read only memory (EEPROM); flash memory; optical or holographic media; magnetic cassettes, magnetic tape, magnetic disk storage or other magnetic storage devices; data transmissions; and/or any other medium that can be used to store information and can be accessed by a computing device. In instances, the memory 530 stores computer-executable instructions 570 for causing the processor 520 to implement aspects of instances of components discussed herein and/or to perform aspects of instances of methods and procedures discussed herein. The memory 530 can comprise a non-transitory computer readable medium storin the computer-executable instructions 570.

The computer-executable instructions 570 may include, for example, computer code, machine-useable instructions, and the like such as, for example, program components capable of being executed by one or more processors 520 (e.g., microprocessors) associated with the computing device 500. Program components may be programmed using any number of different programming environments, including various languages, development kits, frameworks, and/or the like. Some or all of the functionality contemplated herein may also, or alternatively, be implemented in hardware and/or firmware.

According to instances, for example, the instructions 570 may be configured to be executed by the processor 520 and, upon execution, to cause the processor 520 to perform certain processes. In certain instances, the processor 520, memory 530, and instructions 570 are part of a controller such as an application specific integrated circuit (ASIC), field-programmable gate array (FPGA), and/or the like. Such devices can be used to carry out the functions and steps described herein.

The I/O component 550 may include a presentation component configured to present information to a user such as, for example, a display device, a speaker, a printing device, and/or the like, and/or an input component such as, for example, a microphone, a joystick, a satellite dish, a scanner, a printer, a wireless device, a keyboard, a pen, a voice input device, a touch input device, a touch-screen device, an interactive display device, a mouse, and/or the like.

The devices and systems described herein can be communicatively coupled via a network, which may include a local area network (LAN), a wide area network (WAN), a cellular data network, via the internet using an internet service provider, and the like.

Aspects of the present disclosure are described with reference to flowchart illustrations and/or block diagrams of methods, devices, systems and computer program products. It will be understood that each block of the flowchart illustrations and/or block diagrams, and combinations of blocks in the flowchart illustrations and/or block diagrams, can be implemented by computer program instructions.

Various modifications and additions can be made to the exemplary instances discussed without departing from the scope of the disclosed subject matter. For example, while the instances described above refer to particular features, the scope of this disclosure also includes instances having different combinations of features and instances that do not include all of the described features. Accordingly, the scope of the disclosed subject matter is intended to embrace all such alternatives, modifications, and variations as fall within the scope of the claims, together with all equivalents thereof.

We claim:

1. A method comprising:
associating, using a trained machine learning model and based on electrocardiogram (ECG) data, beats with respective initial beat classifications;
generating, using one or more encoder machine learning models, latent space representations of the ECG data for beats associated with the initial beat classifications; and
associating similar shaped beats with each other based on the latent space representations.

2. The method of claim 1, wherein the one or more encoder machine learning models includes a first encoder machine learning model and a second encoder machine learning model, wherein the initial beat classifications include a first classification and a second classification, wherein the latent space representations include first latent space representations for beats associated with the first classification and second latent space representations for beats associated with the second classification, the method further comprising:
generating, using the first encoder machine learning model, the first latent space representations; and
generating, using the second encoder machine learning model, the second latent space representations.

3. The method of claim 2, further comprising:
generating, using the trained machine learning model, individual clips of the ECG data, the individual clips each representing a separate beat.

4. The method of claim 3, wherein the individual clips comprise an individual T-wave for each beat.

5. The method of claim 3, further comprising:
generating the first latent space representations based on the individual clips associated with the first classification; and
generating the second latent space representations based on the individual clips associated with the second classification.

6. The method of claim 3, wherein the first latent space representations include 8-15 datapoints for each beat associated with the first classification, wherein the second latent space representations include 8-15 datapoints for each beat associated with the second classification.

7. The method of claim 2, wherein the first classification is a normal beat classification, wherein the second classification is a ventricular beat classification, wherein the initial beat classifications further include a supraventricular beat classification.

8. The method of claim 1, wherein the ECG data of each beat comprises a first number of datapoints, wherein a number of datapoints of the latent space representations comprises 1-2% of the first number of datapoints.

9. The method of claim 1, wherein the associating similar shaped beats includes grouping the similar shaped beats using a k-means clustering algorithm.

10. The method of claim 1, wherein the associating similar shaped beats includes assigning each group of similar shaped beats a same value.

11. The method of claim 10, further comprising:
receiving—by a computing system—the ECG data, the initial beat classifications, and the values assigned to the beats;
displaying the ECG data in a user interface (UI);
receiving a command to change at least some of the initial beat classifications to a subsequent beat classifications; and
automatically modifying the values to a different value associated with the subsequent beat classifications.

12. A system comprising:
a server comprising:
a first trained machine learning model programmed to generate first latent space representations of beats associated with a first beat classification,
a second trained machine learning model programmed to generate second latent space representations of beats associated with a second beat classification,
a third trained machine learning model programmed to generate third latent space representations of beats associated with a third beat classification, and
one or more processors programmed to apply a clustering algorithm to the first, second, and third latent space representations to associating similar shaped beats with each other.

13. The system of claim 12, wherein electrocardiogram (ECG) data of each beat comprises a first number of datapoints, wherein a number of datapoints of the first, second, and third latent space representations comprises 1-2% of the first number of datapoints.

14. The system of claim 12, wherein the clustering algorithm is a k-means clustering algorithm.

15. The system of claim 12, wherein the first beat classification is a normal beat classification, wherein the second beat classification is a ventricular beat classification, wherein the third beat classification is a supraventricular beat classification.

16. The system of claim 12, wherein the first, second, and third trained machine learning models are neural networks trained using an imbalanced encoder and decoder.

17. The system of claim 12, wherein the first, second, and third trained machine learning models are deep learning neural networks.

18. A method for training an encoder neural network, the method comprising:
inputting data into the encoder neural network, which includes a first number of layers of nodes;
generating latent space representations of the data by the encoder neural network and inputting the latent space representations into a decoder with a second number of layers of nodes, which is less than the first number; and
training the encoder neural network based on the outputs of the decoder responsive to the inputting the latent space representations.

19. The method of claim 18, wherein the data in electrocardiogram (ECG) data, the method further comprising: inputting metadata associated with the ECG data into the encoder neural network.

20. The method of claim 18, wherein the first number is 4-8 times the second number.
